(19) United States
(12) Patent Application Publication
(10) Pub. No.: US 2023/0160293 A1
(43) Pub. Date: May 25, 2023

(54) CONVERSION OF CARBON DIOXIDE CAPTURED FROM FRACTURING OPERATION TO FORMIC ACID USED IN FRACTURING FLUID

(71) Applicant: Halliburton Energy Services, Inc., Houston, TX (US)

(72) Inventors: Philip D. NGUYEN, Houston, TX (US); Tatyana Vladimirov KHAMATNUROVA, Houston, TX (US); Ronald Glen DUSTERHOFT, Houston, TX (US)

(21) Appl. No.: 17/533,472

(22) Filed: Nov. 23, 2021

(51) Int. Cl. E21B 43/34; C25B 3/23; C25B 3/07; B01D 53/62; C09K 8/68; C09K 8/72; C07C 51/10

(52) U.S. Cl. CPC E21B 43/34; C25B 3/23; C25B 3/07; B01D 53/62; C09K 8/68; C09K 8/72; C07C 51/10

(57) ABSTRACT

A method including collecting exhaust gas comprising carbon dioxide (CO₂) at a wellsite to provide a collected exhaust gas, separating CO₂ from the collected exhaust gas to provide a separated CO₂, and forming formic acid utilizing at least a portion of the separated CO₂. At least a portion of the formic acid can be utilizing in a wellbore servicing fluid (e.g., a fracturing fluid) introduced downhole via a wellbore. The exhaust gas can be produced during a wellbore servicing operation at the or another wellbore. A system for carrying out the method is also provided.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD

The present disclosure relates generally to systems and methods of sequestering carbon dioxide (CO₂). More specifically, this disclosure relates to collecting exhaust gas comprising CO₂ and forming formic acid utilizing at least a portion of the CO₂ in the collected exhaust gas. Still more specifically, this disclosure relates to collecting exhaust gas comprising CO₂, separating high purity CO₂ from the collected exhaust gas, forming formic acid utilizing at least a portion of the high purity CO₂, and utilizing the formic acid in a wellbore servicing fluid (e.g., a fracturing fluid).

### BACKGROUND

Natural resources (e.g., oil or gas) residing in a subterranean formation can be recovered by driving resources from the formation into a wellbore using, for example, a pressure gradient that exists between the formation and the wellbore, the force of gravity, displacement of the resources from the formation using a pump or the force of another fluid injected into the well or an adjacent well. A number of wellbore servicing fluids can be utilized during the formation and production from such wellbores. For example, in embodiments, the production of fluid in the formation can be increased by hydraulically fracturing the formation. That is, a treatment fluid (e.g., a fracturing fluid) can be pumped down the wellbore to the formation at a rate and a pressure sufficient to form fractures that extend into the formation, providing additional pathways through which the oil or gas can flow to the well. Subsequently, oil or gas residing in the subterranean formation can be recovered or “produced” from the well by driving the fluid into the well. During production of the oil or gas, substantial quantities of produced water, which can contain high levels of total dissolved solids (TDS), and produced gas can also be produced from the well, and a variety of exhaust gases and flare gases conventionally sent to flare can be formed. For example, oil and gas wells produce oil, gas, and/or byproducts from subterranean formation hydrocarbon reservoirs. A variety of subterranean formation operations are utilized to obtain such hydrocarbons, such as drilling operations, completion operations, stimulation operations, production operations, enhanced recovery operations, and the like. Such subterranean formation operations typically use a large number of vehicles, heavy equipment, and other apparatus (collectively referred to as “machinery” herein) in order to achieve certain job requirements, such as treatment fluid pump rates. Such equipment may include, for example, pump trucks, sand trucks, cranes, conveyance equipment, mixing machinery, and the like. Many of these operations and machinery utilize combustion engines that produce exhaust gases (e.g., including carbon dioxide (CO₂)/greenhouse gas emissions) that are emitted into the atmosphere.

### BRIEF DESCRIPTION OF THE DRAWINGS

For a more complete understanding of this disclosure, reference is now made to the following brief description, taken in connection with the accompanying drawings and detailed description, wherein like reference numerals represent like parts.

FIG. 1 is a schematic flow diagram of a method, according to one or more embodiments of this disclosure;

FIG. 2 is a schematic of a system, according to one or more embodiments of the present disclosure;

FIG. 3 is a schematic of a plurality of machinery that may be located and operated a wellsite for performing a subterranean formation operation and may produce exhaust gas comprising CO₂, according to one or more embodiments of the present disclosure;

FIG. 4 is a schematic of a reaction apparatus, according to one or more embodiments of the present disclosure; and

FIG. 5 is a schematic of a catalytic reaction process suitable for use in an electrocatalytic reaction apparatus, according to one or more embodiments of the present disclosure.

### DETAILED DESCRIPTION

It should be understood at the outset that although an illustrative implementation of one or more embodiments are provided below, the disclosed systems and/or methods can be implemented using any number of techniques, whether currently known or in existence. The disclosure should in no way be limited to the illustrative implementations, drawings, and techniques below, including the exemplary designs and implementations illustrated and described herein, but can be modified within the scope of the appended claims along with their full scope of equivalents.

Carbon dioxide may now be considered a pollutant, and is a product that can be created in significant volumes with oilfield operating equipment, such as hydraulic horsepower pumping units on hydraulic fracturing locations in the field. For example, 150 to 300 metric tons of CO₂ per day per fracturing crew can be produced. Via the system and method of this disclosure, CO₂ emissions can be reduced by the formation of formic acid and the introduction of the formic acid downhole, for example as a component of a wellbore servicing fluid. Accordingly, the system and method described herein enable sequestering of CO₂ and a reduction of greenhouse gas (e.g., CO₂) emissions.

Via the system and method of this disclosure, exhaust gas can be captured at a wellsite (e.g., a hydraulic fracturing location), carbon dioxide separated therefrom, and the separated carbon dioxide utilized to form formic acid. The formic acid can be reintroduced downhole. For example, in embodiments, the formic acid can be incorporated into the hydraulic fracturing fluid and be injected downhole for long term sequestration. Formic acid is formation friendly, and is easier to reinject into a well than carbon dioxide as it requires no compression on location.

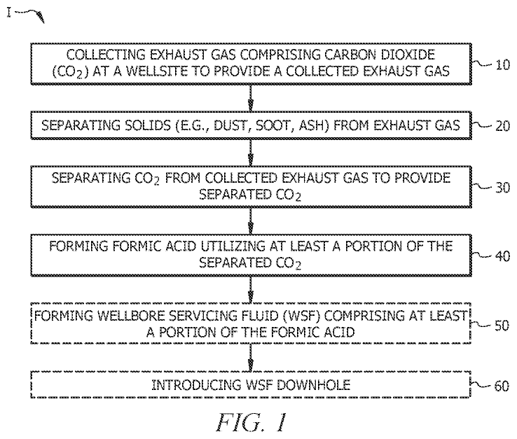

A method of this disclosure will now be described with reference to FIG. 1, which is a schematic flow diagram of a method I according to one or more embodiments of this disclosure. As seen in FIG. 1, method I includes collecting exhaust gas comprising carbon dioxide (CO₂) at a wellsite to provide a collected exhaust gas at 10, separating CO₂ from the collected exhaust gas to provide a separated CO₂ at 30, and forming formic acid utilizing at least a portion of the separated CO₂ at 40. A method I of this disclosure can further comprise separating solids (e.g., dust, soot, ash) from the exhaust gas (e.g., from the produced exhaust gas or the collected exhaust gas) at 20; forming a wellbore servicing fluid (WSF) comprising at least a portion of the formic acid at 50; and/or introducing the WSF downhole at 60. Although depicted in a certain order in FIG. 1, in embodiments, one or more of steps 10 to 60 can be absent, and/or one or more of the steps 10 to 60 can be performed more than once and/or in a different order than described herein or depicted in the embodiment of FIG. 1.

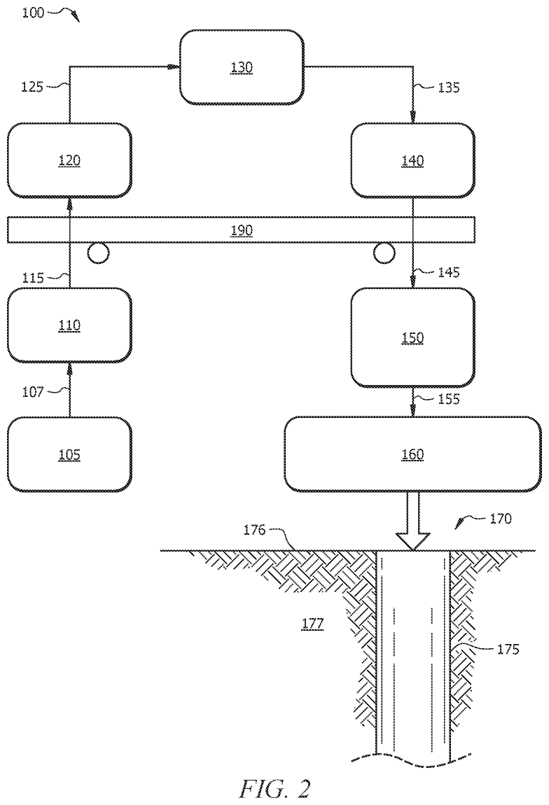

The method of this disclosure will now be detailed and a system for carrying out the method according to embodiments of this disclosure described with reference to FIG. 2, which is a schematic of a system 100 according to one or more embodiments of this disclosure.

With reference now to FIG. 2, system 100 comprises: an exhaust gas collection system 110 configured for collecting exhaust gas comprising carbon dioxide (CO₂) from exhaust gas 107 produced by exhaust gas production equipment or “machinery” 105 at a wellsite 170 to provide a collected exhaust gas 115 (e.g., step 10 of FIG. 1); a CO₂ separation apparatus 130 configured for separating CO₂ from the collected exhaust gas to provide a separated CO₂ 135 (e.g., step 30 of FIG. 1); and a reaction apparatus 140 configured for forming formic acid 145 utilizing at least a portion of the separated CO₂ 135 (e.g., step 40 of FIG. 1). As depicted in FIG. 2, system 100 can further comprise solids removal apparatus 120 configured to separate solids (e.g., soot, dust) from the collected exhaust gas 115 (e.g., step 20 of FIG. 1), WSF production apparatus 150 configured to produce a WSF, wherein the wellbore servicing fluid comprises at least a portion of the formic acid (e.g., step 50 of FIG. 1), and/or pumping apparatus 160 configured for pumping the formic acid 145 and/or wellbore servicing fluid 155 downhole at the wellsite 170 or another wellsite, via a wellbore 175, whereby the formic acid is sequestered downhole (e.g., in a formation, reservoir 177) (e.g., step 60 of FIG. 1).

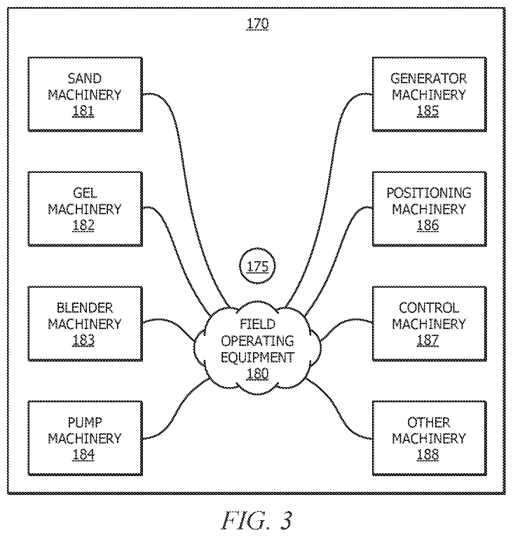

Method I comprises, at 10, collecting exhaust gas comprising CO₂ at a wellsite 170 to provide a collected exhaust gas 115. An exhaust gas collection system 110 can be configured to collect the collected exhaust gas 115 from exhaust gas 107 produced via exhaust gas production equipment 105. Exhaust gas collection system 110 is configured to collect exhaust gas 107 from exhaust gas production equipment 105, such as machinery 180 (FIG. 3, discussed hereinbelow) at a wellsite 170. The field gas operating equipment 180 can comprise one or more vehicles (e.g., diesel trucks, cars, etc.), pumps (e.g., hydraulic pumps, fracturing pumps, etc.), or other equipment at a wellsite 170 that produces an exhaust gas 107 comprising CO₂ from which collected exhaust gas 115 can be collected. Exhaust gas collection apparatus 110 can include piping configured to combine the exhaust gas 107 from a plurality of the exhaust gas production equipment 105 (e.g., machinery 180 of FIG. 3) to provide the collected exhaust gas 115, and introduce the collected exhaust gas 115 to CO₂ separation apparatus 120, storage apparatus to store the collected exhaust gas 115 prior to introduction into CO₂ separation apparatus 130, or a combination thereof. Collecting the collected exhaust gas 115 comprising CO₂ at 10 can be performed by piping exhaust gas 107 from one or more pieces of exhaust gas production equipment 105 (e.g., field machinery 180 at a wellsite 170, described hereinbelow with reference to FIG. 3) together to provide the collected exhaust gas 115.

The exhaust gas comprising CO₂ 107 from which collected exhaust gas 115 is collected in exhaust gas collection system 110 can include greater than or equal to about 0.04, 0.1, 0.5, 1, 2, 3, 4, 5, 6, 7, 8, 9, 10, 20, 30, 40, 50, 60, 70, 80, 90, 95, or 100 volume percent (vol. %) CO₂. By way of examples, the exhaust gas comprising CO₂ 107 can include a waste gas, or one or more components thereof, produced at the wellsite 170 or another jobsite, such as, without limitation, one or more wellsites or industrial plants. The one or more industrial plants can include, without limitation, a cement plant, a chemical processing plant, a mechanical processing plant, a refinery, a steel plant, a power plant (e.g., a gas power plant, a coal power plant, etc.), or a combination and/or a plurality thereof. In embodiments, the exhaust gas comprising CO₂ 115 comprises a waste (or “exhaust”) gas that is a product of fuel combustion, for example, the product of an internal combustion engine, or a gas fired turbine engine, such as, for example, from a microgrid having electric pumps. In embodiments, the internal combustion engine includes an engine fueled by diesel, natural gas (e.g., methane), gasoline, or a combination thereof (e.g., a diesel engine, or a hybrid engine that is fueled by diesel and natural gas). The exhaust gas comprising CO₂ 115 can be produced at the wellsite 170 and/or another jobsite. A plurality of machinery 180 can be located and operated at a wellsite 170 for performing a subterranean formation operation, according to one or more embodiments of the present disclosure, and the exhaust gas comprising CO₂ can, in embodiments, be obtained therefrom. For example, the collected exhaust gas comprising CO₂ 115 can be produced at the wellsite 170 or another wellsite from machinery 180 used to perform a wellbore servicing operation. Such wellbore servicing operations include, without limitation, drilling operations, completion operations, stimulation operations, production operations, enhanced recovery operations, and the like. The machinery may include one or more internal combustion or other suitable engines that consume fuel to perform work at the wellsite 170 and produce exhaust gas comprising CO₂ 107.

The wellbore 175 at wellsite 170 may be a hydrocarbon-producing wellbore (e.g., oil, natural gas, and the like) or another type of wellbore for producing other resources (e.g., mineral exploration, mining, and the like). Machinery 180 typically associated with a subterranean formation operation related to a hydrocarbon producing wellbore, and from which the exhaust gas comprising CO₂ can be produced, can be utilized to perform such operations as, for example, a cementing operation, a fracturing operation, or other suitable operation where equipment is used to drill, complete, produce, enhance production, and/or work over the wellbore. Other surface operations may include, for example, operating or construction of a facility.

As depicted in FIG. 3, which is a schematic of a plurality of machinery 180 that can be included in exhaust gas production equipment 105, the machinery 180 may be located and operated a wellsite 170 for performing a subterranean formation operation and may produce exhaust gas 107 comprising CO₂, according to one or more embodiments of the present disclosure. The machinery from which the exhaust gas comprising CO₂ can be produced, in embodiments, can include sand machinery 181, gel machinery 182, blender machinery 183, pump machinery 184, generator machinery 185, positioning machinery 186, control machinery 187, and/or other machinery 188. The machinery may be, for example, truck, skid or rig-mounted, or otherwise present at the wellsite 170, without departing from the scope of the present disclosure. The sand machinery 181 may include transport trucks or other vehicles for hauling to and storing at the wellsite 170 sand for use in an operation. The gel machinery 182 may include transport trucks or other vehicles for hauling to and storing at the wellsite 170 materials used to make a gelled treatment fluid for use in an operation. The blender machinery 183 may include blenders, or mixers, for blending materials at the wellsite 170 for an operation. The pump machinery 184 may include pump trucks or other vehicles or conveyance equipment for pumping materials down the wellbore 175 for an operation. The generator machinery 185 may include generator trucks or other vehicles or equipment for generating electric power at the wellsite 170 for an operation. The electric power may be used by sensors, control machinery, and/or other machinery. The positioning equipment 186 may include earth movers, cranes, rigs or other equipment to move, locate or position equipment or materials at the wellsite 170 or in the wellbore 175.

The control machinery 187 may include an instrument truck coupled to some, all, or substantially all of the other equipment at the wellsite 170 and/or to remote systems or equipment. The control machinery 187 may be connected by wireline or wirelessly to other equipment to receive data for or during an operation. The data may be received in real-time or otherwise. In another embodiment, data from or for equipment may be keyed into the control machinery.

The control machinery 187 may include a computer system for planning, monitoring, performing or analyzing the job. Such a computer system may be part of a distributed computing system with data sensed, collected, stored, processed and used from, at or by different equipment or locations. The other machinery 188 may include equipment also used at the wellsite 170 to perform an operation.

In other examples, the other machinery 188 may include personal or other vehicles used to transport workers to the wellsite 170 but not directly used at the wellsite 170 for performing an operation.

Many if not most of these various machinery at the wellsite 170 accordingly utilize a diesel or other fuel types to perform their functionality. Such fuel is expended and exhausted as exhaust gas, such as exhaust gas including CO₂. The embodiments described herein provide a system and method for collecting, converting to formic acid, and, in embodiments, introducing the formic acid 145 downhole, thus sequestering CO₂ from such machinery 180 located and operated at a wellsite 170, and reducing atmospheric CO₂ emissions, while reducing material and time costs. It is to be appreciated that other configurations of the wellsite 170 may be employed, without departing from the scope of the present disclosure. Although a number of various machinery 180 at wellsite 170 have been mentioned, many other machinery may utilize diesel or other fuel that creates exhaust gas including CO₂ that may conventionally be exhausted into the atmosphere, but herein utilized to form formic acid as described herein.

In some embodiments, the present disclosure provides collecting exhaust gas including CO₂ 115 from such machinery 180 located and operated at a wellsite 170 and utilizing such collected exhaust gas 115 to form formic acid 145 as detailed herein. In embodiments, exhaust gas 107 is produced by fracturing equipment (e.g., hydraulic fracturing pumping equipment 184, hydraulic horsepower pumping units 184, electrical generation natural gas turbine units 188, electrical generation reciprocating natural gas power units 185, or a combination thereof) utilized to fracture a formation during a fracturing operation in formation 177.

Although described hereinabove with reference to a wellsite 170, a source of the exhaust gas comprising CO₂ 107 that is collected at step 10 of the method I can be any convenient exhaust gas. The exhaust gas source can be a gaseous CO₂ source. This gaseous exhaust gas may vary widely, ranging from air, industrial waste streams, etc. As noted above, the exhaust gas can, in certain instances, include an exhaust waste product from an industrial plant. The nature of the industrial plant may vary in these embodiments, where industrial plants of interest include power plants, chemical processing plants, and other industrial plants that produce exhaust gas comprising CO₂ as a byproduct. By waste stream is meant a stream of gas (or analogous stream) that is produced as a byproduct of an active process of the industrial plant, e.g., an exhaust gas. The gaseous stream may be substantially pure CO₂ or a multi-component gaseous stream that includes CO₂ and one or more additional gases. Multi-component gaseous streams (containing CO₂) that may be employed as a CO₂ source in embodiments of the subject methods include both reducing, e.g., syngas, shifted syngas, natural gas, and hydrogen and the like, and oxidizing condition streams, e.g., flue gases from combustion. Particular multi-component gaseous streams of interest that may be treated according to the subject invention include: oxygen containing combustion power plant flue gas, turbo charged boiler product gas, coal gasification product gas, shifted coal gasification product gas, anaerobic digester product gas, wellhead natural gas stream, reformed natural gas or methane hydrates, and the like.

As noted above, in embodiments, the exhaust gas comprising CO₂ 115 can comprise greater than or equal to about 5, 6, 7, 8, 9, 10, 15, 20, 25, 30, 40, 50, 60, 70, 80, 90, 95, 96, 97, 98, 99, or 100 volume percent (vol %) CO₂. In embodiments, the exhaust gas comprising CO₂ 115 includes primarily CO₂ (e.g., greater than or equal to about 50, 60, 70, 80, 90, 95, 96, 97, 98, 99, or 100 volume percent (vol %) CO₂). For example, when the exhaust gas comprising CO₂ 115 is obtained from a waste gas produced at a different jobsite than the wellsite 170, CO₂ can be separated from the waste gas in order to reduce a volume of gas to be transported to the wellsite 170. For example, when the exhaust gas includes a flue gas from a power plant, which typically contains from about 7 to about 10 vol. % CO₂, the method I can further include transporting the exhaust gas (or a waste gas from which the gas including CO₂ is obtained) from the another jobsite at which the waste gas is obtained to the wellsite 170. In embodiments, the method I can further include separating CO₂ from the waste gas including CO₂ (step 30), prior to transport to wellsite 170, to reduce a volume of gas for transport. Although the separating of the CO₂ from the exhaust gas comprising CO₂ can be performed at the wellsite 170 (e.g., after transport of the waste gas from the another jobsite at which the waste gas is obtained and/or produced to the wellsite 170), to facilitate transportation, the separating of the CO₂ from the exhaust gas comprising CO₂ at 30 can be performed at the another jobsite at which the waste gas is produced and/or obtained and subsequently, the separated CO₂ 135 can be transported to the wellsite 170. Accordingly, CO₂ separation apparatus 130 can be located at a jobsite different from wellsite 170 or can be located at wellsite 170.

As noted above, method I comprises, at 30, separating CO₂ from collected exhaust gas 115. Separating the separated CO₂ 135 from the collected exhaust gas 115 at 30 can comprise separating substantially pure separated CO₂ 135 from the collected exhaust gas 115. That is, in embodiments, the separated CO₂ 135 is substantially pure CO₂. The substantially pure CO₂ (and the substantially pure separated CO₂) can include greater than or equal to about 90, 95, 96, 97, 98, 99, 99.5, 99.8, 99.9, or 100 vol % CO₂. Separating CO₂ from the collected exhaust gas at 30 can comprise passing the collected exhaust gas 115 through a CO₂ separation unit or apparatus 130. CO₂ separation apparatus 130 can comprise any apparatus operable to provide high purity (e.g., greater than or equal to 95, 96, 97, 98, 98.5, 99, 99.5, 99.9, 99.99, or substantially 100 volume percent (vol %) CO₂ from the collected exhaust gas 115 (or a solids-reduced exhaust gas 125 produced in solids removal apparatus 120, described below). CO₂ separation apparatus 130 can operate by separating via amine absorption, calcium oxide (CaO) absorption, filtration, packed bed, another technique, or a combination thereof. In embodiments, CO₂ separation apparatus 130 comprises a membrane unit, an amine unit, a carbon fiber filtration unit, a reaction bed unit, a venturi reactor, batch reactor, continuous reactor, fluidized pack column, another unit configured to remove the at least the portion of the CO₂ from the collected exhaust gas 115, or a combination thereof. In embodiments, the at least the portion of the CO₂ utilized to produce formic acid at step 40 comprises from about 10 to about 90, from about 20 to about 80, from about 30 to about 70, from about 40 to about 60, from about 10 to about 50, from about 50 to about 90, or greater than or equal to about 10, 20, 30, 40, 50, 60, 70, 80, or 90 volume percent (vol %) of the CO₂ in the collected exhaust gas 115.

As noted above, method I can further comprise, at 20, separating solids (e.g., ash, soot, dust) from the exhaust gas (e.g., exhaust gas 107 or collected exhaust gas 115). Separating of the solids can be effected in solids removal apparatus 120 configured to remove solids from a gas. Such gas/solids removal equipment can comprise, for example, a cyclone, a dust filtration unit, a venturi scrubber, carbon fiber filtration unit a bag filtration unit, or a combination thereof.

As noted above, method I further comprises, at 40, forming formic acid utilizing at least a portion of the separated CO₂. A reaction apparatus 140 is configured for forming formic acid from the at least a portion of the separated CO₂ 135. Reaction apparatus 140 can comprise any apparatus operable to produce formic acid from at least a portion of the separated CO₂ 135.

Forming formic acid utilizing at least a portion of the separated CO₂ 135 can comprise electrochemically reacting the at least the portion of the separated CO₂ with water to produce the formic acid and oxygen. In such embodiments, the reaction apparatus 140 can comprise an electrochemical reactor 140A configured for electrochemically reacting the at least the portion of the separated CO₂ 135 with water to produce the formic acid and oxygen.

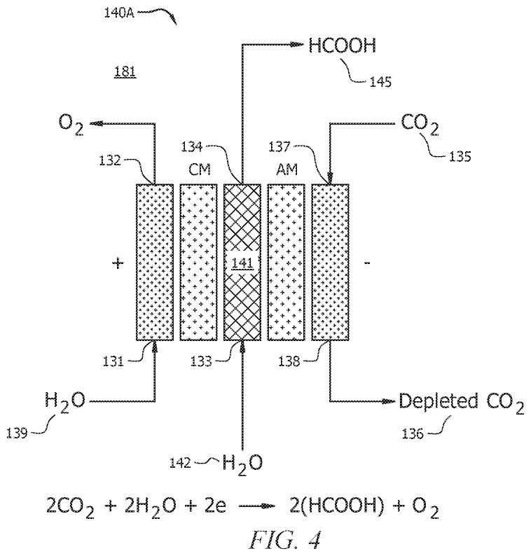

For example, with reference to FIG. 4, which is a schematic of an electrochemical reaction apparatus 140A, according to one or more embodiments of the present disclosure, forming formic acid at 40 can comprise introducing the at least the portion of the separated CO₂ 135 into an electrochemical reaction apparatus 140A. Within electrochemical reaction apparatus 140A, separated CO₂ 135 reacts with water via Equation (1):

2CO₂+H₂O+2e⁻→2(HCOOH)+O₂ Eq. (1),

providing oxygen and a gas 136 depleted in CO₂. Electrochemical reaction apparatus 140A can comprise a cathode side CM and an anode side AM. A DI water 139 inlet 131, O₂ outlet 132, center compartment DI water 142 inlet 133, formic acid HCOOH product 145 outlet 134, CO₂ gas 135 inlet 137 and depleted CO₂ gas 136 product outlet 138 can be located as depicted in FIG. 4. During operation, DI water 139 can be recirculated through the anode flow field using a pump. CO₂ can be humidified (e.g., on the cathode side CM or anode side AM) using a gas humidifier at room temperature (23-25° C.), e.g., using a gas mass flow controller. In a center compartment 141, DI water 142 can be metered into the bottom inlet connection 133 in a single-pass flow mode at a selected flow rate using a pump. The formic acid product solution 145 can be collected from the outlet 134 of the center compartment 141.

Alternatively or additionally, forming formic acid at step 40 can comprise catalytically reacting the at least the portion of the separated CO₂ 135 in water or dimethylsulfoxide (DMSO) to hydrogenate CO₂ and form formic acid. In such embodiments, reaction apparatus 140 can comprise a catalytic reactor configured for forming the formic acid by catalytically reacting the at least the portion of the CO₂ in water or dimethylsulfoxide (DMSO) to hydrogenate CO₂. In such embodiments, reaction apparatus 140 can include a ruthenium (II) catalyst comprising [Ru(PTA)₄Cl₂, wherein PTA=1, 3, 5-triaza-7-phosphaadamantane or variations of thereof:

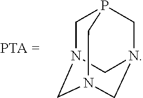

Collecting the collected exhaust gas 115 at 10, separating the separated CO₂ 125 from the collected exhaust gas 115 at 30, and/or forming formic acid 145 at 40 can be performed substantially continuously or intermittently.

As noted above, method I can further comprise forming a WSF comprising at least a portion of the formic acid at 50. Accordingly, system 100 can further include wellbore servicing fluid production apparatus 150 configured to produce a wellbore servicing fluid 155 from at least a portion of the formic acid 145. In embodiments, the wellbore servicing fluid 155 comprises a fracturing fluid, a stimulating fluid, an acidizing fluid, or a combination thereof. In embodiments, the gas exhaust gas collection system 110 is configured to collect at least a portion of the collected exhaust gas 115 from fracturing equipment (e.g., hydraulic fracturing pumping equipment), and the wellbore servicing fluid 155 comprises a fracturing fluid. Although depicted as a disparate unit 150 in FIG. 2, forming the WSF 155 comprising the at least the portion of the formic acid 145 at 50 can comprise injecting the formic acid 145 directly into a wellbore servicing fluid being injected/pumped downhole (e.g., at step 60) to form the WSF 155 comprising the formic acid 145 that is pumped downhole at 60.

As noted hereinabove, method I can further include introducing the WSF downhole (e.g., below a surface 176 of the earth) at 60, whereby formic acid 145 is sequestered downhole (e.g., in a reservoir 177). In such embodiments, system 100 can further include pumping apparatus 150 configured for pumping the wellbore servicing fluid 155 downhole, via a wellbore 175, whereby the formic acid 145 is sequestered downhole (e.g., in a formation, reservoir 177). The formic acid 145 introduced downhole at 50 can be sequestered downhole for a time period of at least 6 months, one year, two years, or five years or more.

The formic acid 145 enables CO₂ to be sequestered long term in the hydrocarbon reservoir 177 as much of the fracturing fluid WSF 155 will remain in the reservoir 177. The formic acid 145 can also react with some formation 177 materials and enhance hydrocarbon mobility.

In embodiments, a method of this disclosure comprises forming formic acid 145 using as a reactant carbon dioxide (CO₂) 135 separated from exhaust gas 107 produced at a wellsite 170 comprising at least one wellbore 175. The method can further comprise introducing the formic acid 145 downhole via the at least one wellbore 175, whereby the formic acid 145 is sequestered downhole (e.g., in a formation, reservoir 177). The formic acid 145 can be introduced downhole as a component of a WSF 155.

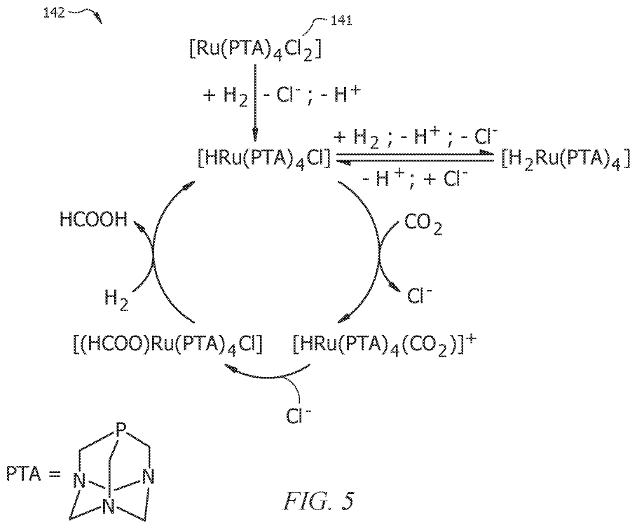

In embodiments, a method of this disclosure comprises collecting/capturing the exhaust gas 115 from hydraulic fracturing pumping equipment on a hydraulic fracturing location at 10, removing unwanted solids, such as dust and soot, from the collected exhaust gas 115 at 20, passing the collected exhaust gas 115 through a CO₂ separation unit 130 (e.g., a membrane unit, an amine unit, a carbon fiber filtration unit, or any other commercial unit capable of removing most of the CO₂ from the collected exhaust gas 115) at 30; introducing the separated CO₂ 135 through an electrolytic or catalytic reaction apparatus 140 with water to convert the CO₂ to formic acid 145 (as described with reference to FIG. 4 and FIG. 5) at 40, and injecting the formic acid 145 directly into a WSF 155 (e.g., a hydraulic fracturing fluid) as it is being pumped downhole at 60.

By way of nonlimiting examples, the WSF 155 comprising formic acid 145 can be introduced downhole to dissolve deposits, break gels, increase a permeability of the formation 177. In embodiments, the WSF 155 comprising at least a portion of the formic acid 145 can be introduced downhole after a perforating stage to establish infectivity by helping to clean up and remove any acid soluble damage in or around a created perforation tunnel that can increase fracture breakdown pressure and fracture treating pressure.

The system and method of this disclosure can provide for continuous, semi-continuous, or intermittent collecting of exhaust gas 115 from exhaust gas production equipment (e.g., machinery 180 at a wellsite 170) and utilization of the collected exhaust gas 115 to produce formic acid 145. The formic acid 145 can be utilized to benefit at the wellsite 170, for example, as a component of a WSF 155 that can be introduced downhole. Accordingly, the herein disclosed system and method enable a reduction in an emission of CO₂ to the atmosphere relative to a method in which formic acid 145 is not formed from the at least the portion of the separated CO₂ 135.

Via the system and method described herein, carbon dioxide can be utilized on location to generate formic acid 145, which can be utilized as part of a hydraulic fracturing fluid WSF 155 that is being injected into a hydrocarbon reservoir 177. Injection of formic acid 145 downhole, as described herein, can be superior to injecting carbon dioxide downhole, as the density of formic acid 145 can be favorably more consistent, and specialized compression equipment that would be required to reinject carbon dioxide downhole may not be needed to inject formic acid 145 downhole. Formic acid 145 is compatible with most hydrocarbon formations 177 and can provide some secondary benefits in helping to improve oil mobility and displacement of oil when the formic acid 145 is introduced downhole.

In embodiments, the system of this disclosure, or one or more components thereof (e.g., exhaust gas collection apparatus 110, solids removal apparatus 120, CO₂ separation apparatus 130, reaction apparatus 140, or a combination thereof) can be provided on a skid (e.g., a trailer skid), whereby at least a portion the separated CO₂ 135 can be converted to formic acid 145 at the wellsite 170. In embodiments, the system of this disclosure, or one or more components thereof (e.g., exhaust gas collection apparatus 110, solids removal apparatus 120, CO₂ separation apparatus 130, reaction apparatus 140, or a combination thereof) is provided as a small-scale formic acid plant (e.g., on one or more skids 190) at wellsite 170, whereby formic acid 145 can be produced on location.

In embodiments, collected exhaust gas 115 is collected on location at wellsite 170, carbon dioxide is separated from the collected exhaust gas 115 on location to provide separated CO₂ 135, the separated carbon dioxide 135 is introduced into a reaction apparatus 140 on location (e.g., at wellsite 170) configured for reaction of the separated CO₂ 135 with water to create formic acid 145, the formic acid 145 is introduced into a WSF 155 (e.g., a hydraulic fracturing fluid, a stimulation fluid), and the WSF 155 is pumped downhole as part of a wellbore operation (e.g., hydraulic fracturing operation, stimulation treatment). The exhaust gas can be produced by equipment/machinery 180 utilized during the wellbore operation (e.g., hydraulic fracturing operation, stimulation treatment), in embodiments.

In embodiments, the system and method of this disclosure enable capture of CO₂ from hydraulic fracturing operations (e.g., via collection of collected exhaust gas 115 from equipment 180 utilized during a hydraulic fracturing operation at 10 and separation of the separated CO₂ 135 from the collected exhaust gas at 30) and sequestration of the separated CO₂ 135 long term (via formation of formic acid 145 from the separated CO₂ at 40 and introduction of the formic acid 145 downhole at 60). The formic acid 145 can be introduced downhole during the hydraulic fracturing operations, in embodiments.

Sequestration of the separated CO₂ 135 via this disclosure can provide for a reduction in the emissions of CO₂ to the atmosphere relative to methods in which the CO₂ is not converted to formic acid 145 and introduced downhole.

### ADDITIONAL DISCLOSURE

The following are non-limiting, specific embodiments in accordance with the present disclosure:

In a first embodiment, a method comprises: collecting exhaust gas comprising carbon dioxide (CO₂) at a wellsite to provide a collected exhaust gas; separating CO₂ from the collected exhaust gas to provide a separated CO₂; and forming formic acid utilizing at least a portion of the separated CO₂.

A second embodiment can include the method of the first embodiment further comprising separating solids (e.g., soot, dust) from the exhaust gas.

A third embodiment can include the method of the first embodiment or the second embodiment further comprising: forming a wellbore servicing fluid comprising at least a portion of the formic acid; and introducing the wellbore servicing fluid downhole via a wellbore, wherein the formic acid is sequestered downhole (e.g., in a reservoir).

A fourth embodiment can include the method of the third embodiment, wherein the wellbore servicing fluid comprises a fracturing fluid, a stimulating fluid, an acidizing fluid, or a combination thereof.

A fifth embodiment can include the method of the third embodiment or the fourth embodiment, wherein the formic acid is sequestered downhole for a time period of at least 6 months, one year, two years, or five years.

A sixth embodiment can include the method of any one of the third to fifth embodiments, wherein the wellbore servicing fluid comprises a fracturing fluid, an acidizing fluid, a stimulating fluid, or a combination thereof.

A seventh embodiment can include the method of any one of the first to sixth embodiments, wherein the exhaust gas is produced by fracturing equipment (e.g., hydraulic fracturing pumping equipment, hydraulic horsepower pumping units, electrical generation natural gas turbine units, electrical generation reciprocating natural gas power units or a combination thereof). An eighth embodiment can include the method of any one of the first to seventh embodiments, wherein forming formic acid comprises electrochemically reacting the at least the portion of the separated CO₂ with water to produce the formic acid and water.

A ninth embodiment can include the method of any one of the first to eighth embodiments, wherein forming the formic acid comprises catalytically reacting the at least the portion of the CO₂ in water or dimethylsulfoxide (DMSO) to hydrogenate CO₂.

A tenth embodiment can include the method of the ninth embodiment, wherein the catalytically reacting comprises hydrogenating the CO₂ in the presence of a ruthenium (II) catalyst comprising [Ru(PTA)₄Cl₂, wherein PTA=1, 3, 5-triaza-7-phosphaadamantane or variations of thereof.

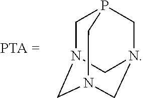

An eleventh embodiment can include the method of any one of the first to tenth embodiments, wherein separating CO₂ from the collected exhaust gas comprises passing the collected exhaust gas through a CO₂ separation unit.

A twelfth embodiment can include the method of the eleventh embodiment, wherein the CO₂ separation unit comprises a membrane unit, an amine unit, a carbon fiber filtration unit, or another unit configured to remove the at least the portion of the CO₂ from the collected exhaust gas.

A thirteenth embodiment can include the method of the twelfth embodiment, wherein the at least the portion of the CO₂ comprises from about 10 to about 90, from about 20 to about 80, from about 30 to about 70, from about 40 to about 60, from about 10 to about 50, from about 50 to about 90, or greater than or equal to about 10, 20, 30, 40, 50, 60, 70, 80, or 90 volume percent (vol %) of the CO₂ in the exhaust gas.

A fourteenth embodiment can include the method of any one of the first to thirteenth embodiments, wherein the method reduces an emission of CO₂ to the atmosphere relative to a method in which formic acid is not formed from the at least the portion of the separated CO₂:

In a fifteenth embodiment, a system comprises: an exhaust gas collection system configured for collecting exhaust gas comprising carbon dioxide (CO₂) at a wellsite to provide a collected exhaust gas; a CO₂ separation apparatus configured for separating CO₂ from the collected exhaust gas to provide a separated CO₂; and a reaction apparatus configured for forming formic acid utilizing at least a portion of the separated CO₂.

A sixteenth embodiment can include the system of the fifteenth embodiment further comprising a solids removal apparatus configured to separate solids (e.g., soot, dust) from the collected exhaust gas.

A seventeenth embodiment can include the system of the fifteenth embodiment or the sixteenth embodiment further comprising wellbore servicing fluid production apparatus configured to produce a wellbore servicing fluid, wherein the wellbore servicing fluid comprises at least a portion of the formic acid.

An eighteenth embodiment can include the system of the seventeenth embodiment further comprising pumping apparatus configured for pumping the wellbore servicing fluid downhole, via a wellbore, whereby the formic acid is sequestered downhole (e.g., in a formation, reservoir).

A nineteenth embodiment can include the system of the eighteenth embodiment, wherein the wellbore servicing fluid comprises a fracturing fluid, a stimulating fluid, an acidizing fluid, or a combination thereof.

A twentieth embodiment can include the system of the eighteenth embodiment or the nineteenth embodiment, wherein the gas exhaust gas collection system is configured to collect at least a portion of the collected exhaust gas from fracturing equipment (e.g., hydraulic fracturing pumping equipment), and wherein the wellbore servicing fluid comprises a fracturing fluid.

A twenty first embodiment can include the system of the twentieth embodiment, wherein the fracturing fluid is introduced downhole via the wellbore by the pumping apparatus (e.g., hydraulic fracturing pumping apparatus).

A twenty second embodiment can include the system of any one of the fifteenth to twenty first embodiments, wherein the gas exhaust gas collection system is configured to collect at least a portion of the collected exhaust gas from fracturing equipment (e.g., hydraulic fracturing pumping equipment).

A twenty third embodiment can include the system of any one of the fifteenth to twenty second embodiments, wherein the reaction apparatus comprises an electrochemical reactor configured for electrochemically reacting the at least the portion of the separated CO₂ with water to produce the formic acid and water.

A twenty fourth embodiment can include the system of any one of the fifteenth to twenty third embodiments, wherein the reaction apparatus comprises a catalytic reactor configured for forming the formic acid comprises catalytically reacting the at least the portion of the CO₂ in water or dimethylsulfoxide (DMSO) to hydrogenate CO₂.

A twenty fifth embodiment can include the system of the twenty fourth embodiment, wherein the catalytic reactor comprises a ruthenium (II) catalyst, e.g., comprising [Ru(PTA)₄Cl₂, wherein PTA=1, 3, 5-triaza-7-phosphaadamantane:

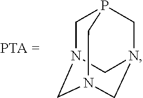

that catalyzes the hydrogenation of the at least the portion of the CO₂.

A twenty sixth embodiment can include the system of any one of the fifteenth to twenty fifth embodiments, wherein the CO₂ separation apparatus comprises a membrane unit, an amine unit, a carbon fiber filtration unit, or another unit configured to remove the at least the portion of the CO₂ from the collected exhaust gas.

A twenty seventh embodiment can include the system of the twenty sixth embodiment, wherein the at least the portion of the CO₂ comprises from about 10 to about 90, from about 20 to about 80, from about 30 to about 70, from about 40 to about 60, from about 10 to about 50, from about 50 to about 90, or greater than or equal to about 10, 20, 30, 40, 50, 60, 70, 80, or 90 volume percent (vol %) of the CO₂ in the exhaust gas.

In a twenty eighth embodiment, a method comprises: producing formic acid using as a reactant carbon dioxide (CO₂) separated from exhaust gas produced at a wellsite comprising at least one wellbore.

A twenty ninth embodiment can include the method of the twenty eighth embodiment further comprising introducing the formic acid downhole via the at least one wellbore, whereby the formic acid is sequestered downhole (e.g., in a formation, reservoir).

A thirtieth embodiment can include the method of the twenty eighth embodiment or the twenty ninth embodiment, wherein the producing is performed substantially continuously or intermittently.

While embodiments have been shown and described, modifications thereof can be made by one skilled in the art without departing from the spirit and teachings of this disclosure. The embodiments described herein are exemplary only, and are not intended to be limiting. Many variations and modifications of the embodiments disclosed herein are possible and are within the scope of this disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). For example, whenever a numerical range with a lower limit, R1, and an upper limit, Ru, is disclosed, any number falling within the range is specifically disclosed. In particular, the following numbers within the range are specifically disclosed: R=R1+k*(Ru—R1), wherein k is a variable ranging from 1 percent to 100 percent with a 1 percent increment, i.e., k is 1 percent, 2 percent, 3 percent, 4 percent, 5 percent, . . . 50 percent, 51 percent, 52 percent, . . . , 95 percent, 96 percent, 97 percent, 98 percent, 99 percent, or 100 percent. Moreover, any numerical range defined by two R numbers as defined in the above is also specifically disclosed. Use of broader terms such as comprises, includes, having, etc. should be understood to provide support for narrower terms such as consisting of, consisting essentially of, comprised substantially of, etc. When a feature is described as “optional,” both embodiments with this feature and embodiments without this feature are disclosed. Similarly, the present disclosure contemplates embodiments where this “optional” feature is required and embodiments where this feature is specifically excluded.

Accordingly, the scope of protection is not limited by the description set out above but is only limited by the claims which follow, that scope including all equivalents of the subject matter of the claims. Each and every claim is incorporated into the specification as embodiments of the present disclosure. Thus, the claims are a further description and are an addition to the embodiments of the present disclosure. The discussion of a reference herein is not an admission that it is prior art, especially any reference that can have a publication date after the priority date of this application. The disclosures of all patents, patent applications, and publications cited herein are hereby incorporated by reference, to the extent that they provide exemplary, procedural, or other details supplementary to those set forth herein.

1. A method comprising:
collecting exhaust gas comprising carbon dioxide (CO₂) at a wellsite to provide a collected exhaust gas;
separating CO₂ from the collected exhaust gas, at the wellsite, to provide a separated CO₂;
forming, at the wellsite, formic acid utilizing at least a portion of the separated CO₂;
forming, at the wellsite, a wellbore servicing fluid comprising at least a portion of the formic acid; and
sequestering the at least the portion of the formic acid downhole by placing the wellbore servicing fluid downhole via a wellbore at the wellsite, wherein CO₂ emissions at the wellsite are reduced relative to a method that does not comprise forming the formic acid from the at least the portion of the separated CO₂ and the placing of the wellbore servicing fluid comprising the at least the portion of the formic acid downhole, and wherein the at least the portion of the formic acid is sequestered downhole long term, wherein long term comprises a time period of at least 6 months.

2. The method of claim 1 further comprising separating solids from the exhaust gas.

3. (canceled)

4. The method of claim 1, wherein the wellbore servicing fluid is a fracturing fluid, a stimulating fluid, an acidizing fluid, or a combination thereof.

5. (canceled)

6. The method of claim 1, wherein the exhaust gas is produced by fracturing equipment.

7. The method of claim 1, wherein forming formic acid comprises electrochemically reacting the at least the portion of the separated CO₂ with water to produce the formic acid and water.

8. The method of claim 1, wherein forming the formic acid comprises catalytically reacting the at least the portion of the CO₂ in water or dimethylsulfoxide (DMSO) to hydrogenate CO₂.

9. The method of claim 1, wherein separating CO₂ from the collected exhaust gas comprises passing the collected exhaust gas through a CO₂ separation unit.

10. A system comprising:
an exhaust gas collection system configured for collecting exhaust gas comprising carbon dioxide (CO₂) at a wellsite to provide a collected exhaust gas;
a CO₂ separation apparatus, at the wellsite, configured for separating CO₂ from the collected exhaust gas to provide a separated CO₂;
a reaction apparatus, at the wellsite, configured for forming formic acid utilizing at least a portion of the separated CO₂;
wellbore servicing fluid production apparatus configured to produce a wellbore servicing fluid, at the wellsite, wherein the wellbore servicing fluid comprises at least a portion of the formic acid; and
pumping apparatus, at the wellsite, configured for sequestering the at least the portion of the formic acid downhole by pumping the wellbore servicing fluid downhole, via a wellbore, wherein CO₂ emissions at the wellsite are reduced relative to a system that is not configured for the forming of formic acid from the CO₂, and the pumping of the wellbore servicing fluid comprising the at least the portion of the formic acid downhole, and wherein the at least the portion of the formic acid is sequestered downhole long term, wherein long term comprises a time period of at least 6 months.

11. The system of claim 10 further comprising a solids removal apparatus configured to separate solids from the collected exhaust gas.

12-13. (canceled)

14. The system of claim 13, wherein the wellbore servicing fluid comprises a fracturing fluid, a stimulating fluid, an acidizing fluid, or a combination thereof.

15. The system of claim 13, wherein the gas exhaust gas collection system is configured to collect at least a portion of the collected exhaust gas from fracturing equipment, and wherein the wellbore servicing fluid comprises a fracturing fluid.

16. The system of claim 10, wherein the gas exhaust gas collection system is configured to collect at least a portion of the collected exhaust gas from fracturing equipment.

17. The system of claim 10, wherein the reaction apparatus comprises an electrochemical reactor configured for electrochemically reacting the at least the portion of the separated CO₂ with water to produce the formic acid and water; and/or a catalytic reactor configured for forming the formic acid by catalytically reacting the at least the portion of the CO₂ in water or dimethylsulfoxide (DMSO) to hydrogenate CO₂.

18. A method comprising:
producing formic acid using as a reactant carbon dioxide (CO₂) separated from exhaust gas produced at a wellsite comprising at least one wellbore; and
sequestering at least a portion of the formic acid downhole by introducing the at least the portion of the formic acid downhole via the at least one wellbore, wherein CO₂ emissions at the wellsite are reduced relative to a method that does not comprise forming the formic acid from the CO₂ separated from the exhaust gas produced at the wellsite and introducing the at least the portion of the formic acid downhole, and wherein the at least the portion of the formic acid is sequestered downhole long term, wherein long term comprises a time period of at least 6 months.

19. (canceled)

20. The method of claim 18, wherein the producing is performed substantially continuously or intermittently.

21. (canceled)

22. The method of claim 7, wherein forming the formic acid comprises electrochemically reacting the at least the portion of the separated CO₂ with water in an electrochemical reaction apparatus, and wherein the electrochemical reaction apparatus comprises a cathode side and an anode side on either side of a central compartment, and wherein the central compartment comprises an inlet for at least a portion of the water and an outlet for the formic acid.

23. (canceled)

24. The system of claim 17, wherein the reaction apparatus comprises an electrochemical reaction apparatus, and wherein the electrochemical reaction apparatus comprises a cathode side and an anode side on either side of a central compartment, and wherein the central compartment comprises an inlet for at least a portion of the water and an outlet for the formic acid.

25. The method of claim 18, wherein the exhaust gas is a product of hydraulic fracturing operations, and wherein the at least the portion of the formic acid is sequestered downhole by introduction into the wellbore as a component of a hydraulic fracturing fluid.

26. The method of claim 24, wherein the cathode side comprises an inlet for a humidified CO₂, wherein the humidified CO₂ comprises the at least the portion of the CO₂ after humidification thereof.

27. The method of claim 22, wherein the cathode side comprises an inlet for a humidified CO₂, wherein the humidified CO₂ comprises the at least the portion of the CO₂ after humidification thereof.
